(19) United States
(12) Patent Application Publication
(10) Pub. No.: US 2012/0135032 A1
(43) Pub. Date: May 31, 2012

(54) GENERATION OF A BROAD T-CELL RESPONSE IN HUMANS AGAINST HIV

(75) Inventors: Paul Chaplin, Grafelfing (DE); Richard Nichols, Munich (DE)

(73) Assignee: BAVARIAN NORDIC A/S, Kvistgaard (DK)

(21) Appl. No.: 13/389,411

(22) PCT Filed: Oct. 6, 2010
(86) PCT No.: PCT/EP2010/006114

§ 371 Date: Feb. 7, 2012

Related U.S. Application Data
(60) Provisional application No. 61/249,644, filed on Oct. 8, 2009.

(30) Foreign Application Priority Data
Feb. 8, 2010 (EP) 10001264.0

(51) Int. Cl. A61K 39/21; A61P 31/18

(52) U.S. Cl. 424/199.1

(57) ABSTRACT

The present invention relates to a recombinant Modified Vaccinia virus Ankara (MVA) comprising in the viral genome one or more expression cassettes for the expression of HIV proteins selected from Gag, Pol, Tat, Vif, Vpu, Vpr, Rev and Nef or a part or a derivative thereof or selected from Gag, Pol, Vpu, Vpr, Rev and Nef or a part or a derivative thereof for use as medicament or vaccine and its use for the treatment and/or prevention of HIV infections and AIDS.

The present invention relates to a recombinant Modified Vaccinia virus Ankara (MVA) comprising in the viral genome one or more expression cassettes for the expression of HIV proteins selected from Gag, Pol, Tat, Vif, Vpu, Vpr, Rev and Nef or parts or derivatives thereof for use as medicament or vaccine and its use for the treatment and/or prevention of HIV infections and AIDS.

### BACKGROUND OF THE INVENTION

The Human Immunodeficiency virus (HIV) is the causative agent of the Acquired Immunodeficiency Syndrome (AIDS). Like all retroviruses the genome of the virus encodes the Gag, Pol and Env proteins. In addition, the viral genome encodes further regulatory proteins, i.e. Tat and Rev, as well as accessory proteins, i.e. Vpr, Vpx, Vpu, Vif and Nef.

Despite public health efforts to control the spread of the AIDS epidemic, the number of new infections is still increasing. The World Health Organization estimated the global epidemic at 37.8 million infected individuals at the end of the year 2003. Without further improvements on comprehensive prevention mechanisms, the number of new HIV infections to occur, globally, this decade is projected to be 45 million (2004 Report on The Global AIDS Epidemic, UNAIDS and WHO).

HIV infection is a chronic infectious disease that can be partially controlled, but not yet cured. There are effective means of preventing complications and delaying progression to AIDS. At the present time, not all persons infected with HIV have progressed to AIDS, but it is generally believed that the majority will.

A combination of several antiretroviral agents, termed Highly Active Anti-Retroviral Therapy (HAART), has been highly effective in reducing viral load, which can improve T-cell counts. This is not a cure for HIV, and people on HAART with suppressed levels of HIV can still transmit the virus to others. However, there is good evidence that if the levels of HIV remain suppressed and the CD4 count remains greater than 200, then the quality and length of life can be significantly improved and prolonged. Given the steady spread of the epidemic, a number of different HIV vaccine delivery strategies such as novel vectors or adjuvant systems have now been developed and evaluated in different pre-clinical settings as well as in clinical trials. The first vaccine candidate that entered a phase-III clinical trial is based on envelope gp 120 protein in alum (Francis et al., AIDS Res. Hum. Retroviruses 1998; 14 (Suppl 3)(5): S325-31). However, the results of the first clinical studies were not very promising.

Although drugs used in HAART regimens are able to reduce the viral titres, there are several concerns about antiretroviral regimens. One main problem concerning HAART is (long-term) side effects and thereby compliance of the patient. If patients miss doses, drug resistance can develop. Also, anti-retroviral drugs are costly, and the majority of the world's infected individuals do not have access to medications and treatments for HIV and AIDS.

The vaccines that were tested for efficacy in the past are usually based on single HIV proteins such as Env. However, even if an immune response was generated against such a single protein, e.g. Env, said immune response proved not to be effective. One reason for the ineffectiveness is the high mutation rate of HIV, in particular with respect to the Env protein resulting in viruses the proteins of which are not recognized by the immune response induced by the vaccine.

Since no effective prophylactic treatment is available there is still a need to bring an effective vaccine to the clinic.

### DETAILED DESCRIPTION OF THE INVENTION

The present inventors have surprisingly found that an MVA-based HIV vaccine which comprises the HIV-1 proteins Gag, Pol, Tat, Vif, Vpu, Vpr, Rev and Nef is capable of inducing a T-cell response in humans to up to six of these HIV proteins, in particular in immunocompromised humans who are HIV-infected. This finding could not have been expected, since it is known that MVA as such provides immunodominant epitopes to the immune system such that epitopes to which a T-cell response is desired are, so to say, overlaid by these immunodominant epitopes. Moreover, it was observed during a clinical trial that the immune responses of the vaccinated HIV-infected humans was still higher than baseline 20 weeks after receiving the first immunization.

Indeed, in contrast to the present invention, no clinical trial in humans, but either in vitro data or in vivo data in a mouse model were generated in order to investigate an effect of an HIV-vaccine on immune cells.

In fact, animal studies are not as good as humans since the models do not often predict human response rates and magnitudes of responses. All the more mice and rabbits are more immunologically responsive as humans. Accordingly, the data acquired by the present inventors have to be valuated highly and could not have been expected in view of the known phenomenon of immunodominance by MVA, the less so because they were acquired from humans infected with HIV. The HIV-infected humans that participated at the clinical trial described in the appended Examples had a CD4 count of less than 350/μl and based on this low number one would not have expected such a broad T cell response against six of the eight HIV-1 proteins comprised by the MVA-based vaccine administered to the participants of the clinical trial.

For example, US 2006/188961 and WO 03/097675 described MVA-based HIV vaccines. However, these documents do not reveal a broad T-cell response against HIV-1 proteins as applied in the recombinant MVA as described herein.

EP 1921146 and WO 01/47955 describe an MVA-based HIV vaccine comprising CTL epitopes of Gag, Pol and Nef or Gag, Pol, Nef, Vpr and Vpu for the induction of a T-cell response. However, these documents fails to provide data showing a broad T-cell response, let alone data acquired in humans.

WO 2006/123256 is quite similar to WO 01/47955 and fails to provide anything that goes beyond what WO 01/47955 describes, apart from more specific CTL epitopes.

WO 2008/118936 describes an MVA-based HIV vaccine comprising an HIV-protein selected from Env, Gag, Nef, RT, Tat and Rev. Yet, this document suffers from the shortcoming of merely having animal model data which cannot be reasonably extrapolated to humans.

Greenough et al. (2008), Vaccine. 26: 6883-6893 reports about safety and immunogenicity studies of a recombinant poxvirus HIV-1 vaccine comprising Env, Tat, Rev, Nef and RT which is administered to young adults on HAART, thus, being infected with HIV. However, the authors report that their vaccine is not likely to move forward in development.

However, to this end, one would not have expected a broad T cell response against six out of eight HIV proteins, let alone such an immune response in immunocompromised subjects. In particular, neither in vitro data nor animal models can form the basis for a reasonable expectation of success, in particular in the field of HIV treatment. This is so because the “real” model system for HIV is a human, in particular, an HIV infected humans.

Based on the animal experiments and the attempts to even multiply an immune response by lining up multiple CTL epitopes (such as in WO 01/47955 or WO 2008/118936) which did not necessarily result in the generation of an immune response against the desired HIV proteins, it could not have been expected that in immunocompromised humans six out of eight HIV proteins are recognized by the immune system.

Accordingly, the present invention relates to a recombinant Modified Vaccinia virus Ankara (MVA) comprising in the viral genome one or more expression cassettes for the expression of at least three HIV proteins selected from Gag, Pol, Tat, Vif, Vpu, Vpr, Rev and Nef or parts or derivatives thereof for use as medicament or vaccine.

In one embodiment said recombinant MVA comprises in the viral genome one or more expression cassettes for the expression of at least six HIV proteins selected from Gag, Pol, Tat, Vif, Vpu, Vpr, Rev and Nef or parts or derivatives thereof for use as medicament or vaccine.

In another embodiment said recombinant MVA comprises in the viral genome one or more expression cassettes for the expression of at least eight HIV proteins selected from Gag, Pol, Tat, Vif, Vpu, Vpr, Rev and Nef or parts or derivatives thereof for use as medicament or vaccine.

In further specific embodiments of the present invention said recombinant MVA comprises in the viral genome one or more expression cassettes for the expression of three or four or five or six or seven or eight HIV proteins selected from Gag, Pol, Tat, Vif, Vpu, Vpr, Rev and Nef or parts or derivatives thereof for use as medicament or vaccine.

The present invention also encompasses a recombinant Modified Vaccinia virus Ankara (MVA) comprising in the viral genome one or more expression cassettes for the expression of eight HIV proteins selected from Gag, Pol, Tat, Vif, Vpu, Vpr, Rev and Nef and one or more additional structural and/or accessory/regulatory HIV proteins or parts or derivatives thereof for use as medicament or vaccine.

In a preferred embodiment the present invention relates to a recombinant Modified Vaccinia virus Ankara (MVA) comprising in the viral genome one or more expression cassettes for the expression of the HIV proteins Gag, Pol, Tat, Vif, Vpu, Vpr, Rev and Nef or parts or derivatives of said proteins for use as medicament or vaccine.

In a further preferred embodiment the present invention relates to a recombinant Modified Vaccinia virus Ankara (MVA) comprising in the viral genome one or more expression cassettes for the expression of the HIV proteins Gag, Pol, Vpu, Vpr, Rev and Nef or a part or a derivative of said proteins for use as medicament or vaccine.

In a particular preferred embodiment the present invention relates to a recombinant Modified Vaccinia virus Ankara (MVA) comprising in the viral genome one or more expression cassettes for the expression of at least six HIV proteins selected from Gag, Pol, Tat, Vif, Vpu, Vpr, Rev and Nef or a part thereof or a derivative of said proteins for use in inducing a T-cell response to at least three of the HIV proteins selected from Gag, Pol, Vpr, Vpu, Rev and Nef in a human subject.

In another particular preferred embodiment the present invention relates to a recombinant Modified Vaccinia virus Ankara (MVA) comprising in the viral genome one or more expression cassettes for the expression of at least six HIV proteins selected from Gag, Pol, Vpu, Vpr, Rev and Nef or parts thereof or derivatives of said proteins for use in inducing a T-cell response to at least three of the HIV proteins selected from Gag, Pol, Vpr, Vpu, Rev and Nef in a human subject.

In a further particular preferred embodiment the present invention relates to a recombinant Modified Vaccinia virus Ankara (MVA) comprising in the viral genome one or more expression cassettes for the expression of at least eight HIV proteins selected from Gag, Pol, Tat, Vif, Vpu, Vpr, Rev and Nef or a part thereof or a derivative of said proteins for use in inducing a T-cell response to at least three of the HIV proteins selected from Gag, Pol, Vpr, Vpu, Rev and Nef in a human subject.

The present invention also provides a pharmaceutical or vaccine composition comprising a recombinant MVA as defined herein, in particular a recombinant MVA comprising in the viral genome one or more expression cassettes for the expression of at least six HIV proteins selected from Gag, Pol, Tat, Vif, Vpu, Vpr, Rev and Nef or parts thereof or a derivative of said proteins.

More preferably, a pharmaceutical or vaccine composition of the present invention comprises a recombinant MVA comprising in the viral genome one or more expression cassettes for the expression of at least eight HIV proteins selected from Gag, Pol, Tat, Vif, Vpu, Vpr, Rev and Nef or a part thereof or derivatives of said proteins.

In a further preferred embodiment the composition comprises a recombinant MVA comprising in the viral genome one or more expression cassettes for the expression of at least six HIV proteins selected from Gag, Pol, Vpu, Vpr, Rev and Nef or parts thereof or derivatives of said proteins.

In a preferred embodiment, the MVA comprised in the pharmaceutical or vaccine composition is at a dosage of about 2×10 TCID₅₀/ml in said pharmaceutical or vaccine composition.

In another preferred embodiment, the MVA comprised in the pharmaceutical or vaccine composition is prepared for being administered at three time intervals. The three time intervals are preferably at week 0, 4 and 12.

With the recombinant MVA according to the present invention it is now possible to express numerous HIV proteins. These numerous proteins are capable to induce a wide range of immune responses. Thus, the likelihood is increased that a protective immune response is generated that is effective against different HIV isolates.

According to the present invention Modified Vaccinia virus Ankara (MVA) is suitable for use in humans and several animal species such as mice and non-human primates. MVA is known to be exceptionally safe.

The term “subject” when used herein refers in particular to a human subject. A human subject when referred to herein may be immunocompromised, for example, due to infection with HIV, i.e., the human subject is HIV-infected, for example, infected with HIV-1. The human subject as referred to herein may be characterized in that it has a CD4 cell count of less than 350/μl.

“Immunocompromised” when used herein is a state in which the immune system's ability to defend or fight infectious disease is compromised or entirely absent.

MVA has been generated by long-term serial passages of the Ankara strain of Vaccinia virus (CVA) on chicken embryo fibroblasts (for review see Mayr, A., Hochstein-Mintzel, V. and Stickl, H. [1975] Infection 3, 6-14; Swiss Patent No. 568, 392). Examples for MVA virus strains that have been deposited in compliance with the requirements of the Budapest Treaty and that are useful for the generation of recombinant viruses according to the present invention are strains MVA 572 deposited at the European Collection of Animal Cell Cultures (ECACC), Salisbury (UK) with the deposition number ECACC 94012707 on Jan. 27, 1994; MVA 575 deposited under ECACC 00120707 on Dec. 7, 2000; and MVA-BN deposited with the number 00083008 at the ECACC on Aug. 30, 2000.

Several excellent properties of the MVA strain pertinent to its use in vaccine development have been demonstrated in extensive clinical trials (Mayr et al., Zbl. Bakt. Hyg. I, Abt. Org. B 167, 375-390 [1987]). During these studies, performed in over 120,000 humans, including high-risk patients, no side effects were seen (Stickl et al., Dtsch. med. Wschr. 99, 2386-2392 [1974]).

It has been further found that MVA is blocked in the late stage of the virus replication cycle in mammalian cells (Sutter, G. and Moss, B., Proc. Natl. Acad. Sci. USA 89, 10847-10851 [1992]). Accordingly, MVA fully replicates its DNA, synthesizes early, intermediate, and late gene products, but is not able to assemble mature infectious virions, which could be released from an infected cell. For this reason, namely, its replication-restricted nature, MVA serves as a gene expression vector.

Therefore, in one embodiment of the present invention the recombinant MVA is selected from MVA strains MVA 575, MVA 572 and MVA-BN.

In a preferred embodiment, the recombinant MVA virus of the invention is replication incompetent in humans and non-human primates. The terms MVA virus that is “replication incompetent” in humans and/or non-human primates, and the synonymous term virus that is “not capable of being replicated to infectious progeny virus” in humans and/or non-human primates, both refer preferably to MVA viruses that do not replicate at all in the cells of the human and/or non-human primate vaccinated with said virus. However, also within the scope of the present application are those viruses that show a minor residual replication activity that is controlled by the immune system of the human and/or non-human primate to which the recombinant MVA virus is administered.

In one embodiment, the replication incompetent recombinant MVA viruses may be viruses that are capable of infecting cells of the human and/or non-human primate in which the virus is used as vaccine. Viruses that are “capable of infecting cells” are viruses that are capable of interacting with the host cells to such an extent that the virus, or at least the viral genome, becomes incorporated into the host cell. Although the viruses used according to the invention are capable of infecting cells of the vaccinated human and/or non human primate, they are not capable of being replicated to infectious progeny virus in the cells of the vaccinated human and/or non-human primate.

According to the invention, it is to be understood, that a virus that is capable of infecting cells of a first animal species, but is not capable of being replicated to infectious progeny virus in said cells, may behave differently in a second animal species. For example,

MVA-BN and its derivatives (see below) are viruses that are capable of infecting cells of the human, but that are not capable of being replicated to infectious progeny virus in human cells. However, the same viruses are efficiently replicated in chickens; i.e., in chickens, MVA-BN is a virus that is both capable of infecting cells and capable of being replicated to infectious progeny virus in those cells.

A suitable test that allows one to predict whether a virus is capable or not capable of being replicated in humans is disclosed in WO 02/42480 (incorporated herein by reference) and uses the severely immune compromised AGR129 mice strain. Furthermore, instead of the AGR129 mice, any other mouse strain can be used that is incapable of producing mature B and T cells, and as such is severely immune compromised and highly susceptible to a replicating virus. The results obtained in this mouse model reportedly are indicative for humans and, thus, according to the present application, a virus that is replication incompetent in said mouse model is regarded as a virus that is “replication incompetent in humans.”

In other embodiments, the viruses according to the invention are preferably capable of being replicated in at least one type of cells of at least one animal species. Thus, it is possible to amplify the virus prior to its administration to the animal that is to be vaccinated and/or treated. By way of example, reference is made to MVA-BN that can be amplified in CEF (chicken embryo fibroblasts) cells, but that is a virus that is not capable of being replicated to infectious progeny virus in humans.

The term “derivatives” or “variant” of a virus according to the invention refers to progeny viruses showing the same characteristic features as the parent virus, but showing differences in one or more parts of its genome. The term “derivative” or “variant of MVA” or “MVA-BN” describes a virus which has the same functional characteristics compared to MVA. For example, a derivative/variant of MVA-BN has the characteristic features of MVA-BN, preferably of the MVA-BN as deposited at ECACC with deposit no. 00083008. One of these characteristics of MVA-BN, or of a variant thereof, is its attenuation and having no capability of reproductive replication in human cell lines, respectively, such as the human keratinocyte cell line HaCaT, the human embryo kidney cell line 293, the human bone osteosarcoma cell line 143 B, and the human cervix adenocarcinoma cell line HeLa.

In addition and/or alternatively, MVA-BN and derivatives have the property of failure to replicate in a mouse model that is incapable of producing mature B and T cells and/or have the ability to induce at least the same level of specific immune response in vaccinia virus prime/vaccinia virus boost regimes when compared to DNA prime/vaccinia virus boost regimes.

A vaccinia virus, in particular an MVA strain, is regarded as inducing at least substantially the same level of immunity in vaccinia virus prime/vaccinia virus boost regimes when compared to DNA-prime/vaccinia virus boost regimes if the CTL response as measured in one or two of the “assay 1” and “assay 2” as disclosed in WO 02/42480 is at least substantially the same in vaccinia virus prime/vaccinia virus boost regimes when compared to DNA-prime/vaccinia virus boost regimes. More preferably the CTL response after vaccinia virus prime/vaccinia virus boost administration is higher in at least one of the assays, when compared to DNA-prime/vaccinia virus boost regimes. Most preferably the CTL response is higher in both assays.

The virus used according to the present invention can be a clone purified virus, such as a monoclonal virus.

The virus used according to the present invention can be a virus that has been produced/passaged under serum free conditions to reduce the risk of infections with agents contained in serum.

MVA according to the present invention is administered in a concentration range of 10⁴ to 10⁹ TCID₅₀/ml, preferably in a concentration range of e.g. 10⁵ to 5×10 ⁸ TCID₅₀/ml, more preferably in a concentration range of e.g. 10⁶ to 10⁸ TCID₅₀/ml or 10⁷ to 10⁹ TCID₅₀/ml, even more preferably in a concentration range of e.g. 10⁸ to 10⁹ TCID₅₀/ml and most preferably at a concentration of 2×10⁸ TCID₅₀/ml. The actual concentration depends on the type of the virus and the animal species to be vaccinated. For MVA-BN a typical vaccination dose for humans comprises 5×10⁷ TCID₅₀ to 5×10⁸ TCID₅₀, such as about 1 or 2×10⁸ TCID₅₀, administered subcutaneously.

In a preferred embodiment of the present invention, the recombinant MVA described herein is administered at least three times when being applied in the uses and methods of the invention.

In another preferred embodiment of the present invention, the recombinant MVA is administered at week 0, 4 and 12 when being applied in the uses and methods of the invention.

It is possible to induce an immune response with a single administration of the recombinant MVA as defined above, in particular with strain MVA-BN and its derivatives. Usually one may use the MVA according to the present invention, in particular MVA-BN and its derivatives in homologous prime boost regimes, i.e. it is possible to use a recombinant MVA for a first vaccination and to boost the immune response generated in the first vaccination by administration of the same or a related recombinant MVA than the one used in the first vaccination. The recombinant MVA according to the present invention, in particular MVA-BN and its derivatives may also be used in heterologous prime-boost regimes in which one or more of the vaccinations is done with an MVA as defined above and in which one or more of the vaccinations is done with another type of vaccine, e.g. another virus vaccine, a protein or a nucleic acid vaccine.

The mode of administration may be intravenously, intramuscularly intradermal, intranasal, or subcutaneously. Preferred is intravenous, intramuscular or, in particular, subcutaneous administration. However, any other mode of administration may be used such as scarification.

The term “HIV” as used in the context of the present application refers to any kind of HIV including HIV-1 and HIV-2 and the corresponding clades such as HIV-1 clade A, B or C. Examples are HIV-1 strains such as strains of clade B.

According to one embodiment of the present invention the HIV proteins encoded by the expression cassettes are HIV-1 proteins.

The term “part of an HIV protein” as used in the present application refers to a peptide or protein comprising at least 10 consecutive amino acids of the corresponding full length HIV protein, such as at least 20, 30 or 40 amino acids of said full length protein. By way of example and without being restricted to said embodiments reference is made to the various HIV sequences as disclosed in the genebank database, in particular to the sequence of the HIV-1 isolate HXB2R having the genebank accession number K03455.

The term “derivative of the amino acid sequence of a HIV protein” as used in the present specification refers to HIV proteins that have an altered amino acid sequence compared to the corresponding naturally occurring HIV protein. An altered amino acid sequence may be a sequence in which one or more amino acids of the sequence of the HIV protein are substituted, inserted or deleted and, thus, mutated. More particularly a “derivative of the amino acid sequence of a HIV protein” is an amino acid sequence showing an identity of at least 50%, such as of at least, 60%, 65%, 70%, 75%, or of at least 80% or 85%, or even of at least 90%, 95%, 98%, or 99% when the amino acid sequence of the protein derivative is compared to the amino acid sequence of the respective HIV protein of known HIV isolates. An amino acid sequence is regarded as having the above indicated sequence homology or identity even if the homology/identity is found for the corresponding protein of only one HIV isolate, irrespective of the fact that there might be corresponding proteins in other isolates showing a lower homology. By way of example, if a Vpr derivative in the fusion protein shows a homology of 95% to the Vpr sequence of one HIV isolate, but only a homology of 50-70% to (all) other HIV isolates, the homology of said Vpr derivative is regarded as being of at least 90%. In particular, the term “derivative of an HIV protein” refers to an amino acid sequence showing a homology of at least 50%, 60%, 65% 70%, 75% 80%, 85% or 90%, 95%, 98%, or 99% to the respective HIV protein in the HIV-1 isolate HXB2R (genebank accession number K03455).

Derivative(s) of HIV proteins and part(s) of an HIV protein can have full activity, reduced activity, no activity, or transdominant activity. For example, the recombinant MVA according to the present invention expresses regulatory and/or accessory proteins of HIV. These proteins have a biological activity that may have undesired side effects. Thus, it is within the scope of the present invention that one or more HIV proteins expressed from the recombinant MVA may have a reduced activity compared to the wild type protein.

Tests are known to the person skilled in the art how to determine whether a HIV protein has reduced biological activity:

The molecular mechanism of the Vif protein, which is essential for viral replication in vivo, remains unknown, but Vif possesses a strong tendency toward self association. This multimerization was shown to be important for Vif function in viral life cycle (Yang S. et al., J Biol Chem 2001; 276: 4889-4893). Additionally vif was shown to be specifically associated with the viral nucleoprotein complex and this might be functionally significant (Khan M. A. et al., J Virol. 2001; 75 (16): 7252-65). Thus, a vif protein with reduced activity shows a reduced multimerization and/or association to the nucleoprotein complex.

The Vpr protein plays an important role in the viral life cycle. Vpr regulates the nuclear import of the viral preintegration complex and facilitates infection of non dividing cells such as macrophages (Agostini et al., AIDS Res Hum Retroviruses 2002; 18(4):283-8). Additionally, it has transactivating activity mediated by interaction with the LTR (Vanitharani R. et al., Virology 2001; 289 (2):334-42). Thus, a Vpr with reduced activity shows decreased or even no transactivation and/or interaction with the viral preintegration complex.

Vpx, which is highly homologous to Vpr, is also critical for efficient viral replication in non-dividing cells. Vpx is packaged in virus particles via an interaction with the p6 domain of the gag precursor polyprotein. Like Vpr Vpx is involved in the transportation of the preintegration complex into the nucleus (Mahalingam et al., J. Virol 2001; 75 (1):362-74). Thus, a Vpx with reduced activity has a decreased ability to associate to the preintegration complex via gag precurser.

The Vpu protein is known to interact with the cytoplasmic tail of the CD4 and causes CD4 degradation (Bour et al., Virology 1995; 69 (3):1510-20). Therefore, Vpu with reduced activity has a reduced ability to trigger CD4 degradation.

The relevant biological activity of the well-characterized Tat protein is the transactivation of transcription via interaction with the transactivation response element (TAR). It was demonstrated that Tat is able to transactivate heterologous promoters lacking HIV sequences other than TAR (Han P. et al., Nucleic Acid Res 1991; 19 (25):7225-9). Thus, a Tat protein with reduced activity shows reduced transactivation of promoters via the TAR element. According to the present invention it is also possible to use a transdominant Tat. The transdominant Tat may be obtained by making the following substitutions: 22 (Cys>Gly) and 37 (Cys>Ser)

Nef protein is essential for viral replication responsible for disease progression by inducing the cell surface downregulation of CD4 (Lou T et al., J Biomed Sci 1997;4(4):132). This downregulation is initiated by direct interaction between CD4 and Nef (Preusser A. et al., Biochem Biophys Res Commun 2002;292 (3):734-40). Thus, Nef protein with reduced function shows reduced interaction with CD4. Examples are Nef proteins that are truncated at the amino terminus such as a protein in which the 19 N terminal amino acids are deleted. According to the present invention it is also possible to use a truncated Nef, in particular in which the 19 N terminal amino acids are deleted.

The relevant function of Rev is the posttranscriptional transactivation initiated by interaction with the Rev-response element (RRE) of viral RNA (Iwai et al., 1992; Nucleic Acids Res 1992; 20 (24):6465-72). Thus, a Rev with reduced activity shows a reduced interaction with the RRE.

According to one embodiment of the present invention one or more of the HIV proteins are expressed as individual proteins.

According to a further embodiment two or more of the HIV proteins are expressed as a fusion protein. Preferably, two or more of the HIV accessory/regulatory proteins are expressed as a fusion protein.

In this context reference is made to WO 03/097675, the content of which is herewith incorporated by reference.

By way of example a recombinant MVA according to the present invention, such as MVA-BN and its derivatives, may express (i) Vif-Vpu-Vpr-Rev as fusion protein in this or a different order, wherein Vif, Vpu, Vpr and Rev stand for full length proteins, or parts or derivatives of the full length proteins (see definition above), (ii) Nef or a part or derivative thereof, in particular a Nef protein in which N-terminal amino acids are deleted (i.e. N-terminal truncated Nef), such as the first 19 amino acids, (iii) Tat or a part or derivative thereof, in particular a transdominant Tat and (iv) Gag-Pol fusion protein, wherein Gag and Pol stand for full length proteins, or parts or derivatives of the full length proteins, arranged in the exemplified order, or in the reverse order, Pol-Gag.

The number of expression cassettes from which the HIV proteins are expressed is not critical. By way of example the HIV proteins may be expressed from 2 to 5 expression cassettes. One expression cassette may express a Vif-Vpu-Vpr-Rev as fusion protein in this or a different order, wherein Vif, Vpu, Vpr and Rev stand for full length proteins, or parts or derivatives of the full length proteins (see definition above), a second expression cassette may express Nef or a part or derivative thereof, in particular a Nef protein in which N-terminal amino acids are deleted, such as the first 19 amino acids, a third expression cassette may express Tat or a part or derivative thereof, in particular a transdominant Tat, and a fourth expression cassette may express a Gag-Pol fusion protein, wherein Gag and Pol stand for full length proteins, or parts or derivatives of the full length proteins, arranged in the exemplified order, or in the reverse order, Pol-Gag. Preferably, the expression cassette coding for the Gag-Pol fusion protein and the expression cassette coding for Tat are inserted into the same insertion site.

The expression of heterologous nucleic acid sequence is preferably, but not exclusively, under the transcriptional control of a poxvirus promoter. An example of a suitable poxvirus promoter is the cowpox ATI promoter (see WO 03/097844). It is possible that the expression of each expression cassette is controlled by a different promoter. Alternatively it is also possible that all expression cassettes are controlled by a copy of the same promoter. By way of example the invention relates to a recombinant virus in which all HIV expression cassettes, such as the four expression cassettes exemplified

WO 2011/042180 ₁₅ PCT/EP2010/006114 above are controlled by a cowpox ATI promoter or derivative thereof as defined in WO 03/097844.

In the recombinant MVA according to the present invention, such as MVA-BN and its derivatives, the expression cassettes may be inserted into 1 to 10 insertion sites in the viral genome.

It was unexpectedly found that recombinant MVA, in particular MVA-BN and its variants for the expression of the HIV proteins or parts or derivatives thereof can be easily obtained if not all expression cassettes are inserted into the same insertion side. Thus, the different expression cassettes may be inserted into 1 to 5, or 2 to 8, or 3 to 5, or into 3 insertion sites in the viral genome.

The insertion of heterologous nucleic acid sequence may be done into a non-essential region of the virus genome. According to another embodiment of the invention, the heterologous nucleic acid sequence is inserted at a naturally occurring deletion site of the MVA genome (disclosed in PCT/EP96/02926). According to a further alternative the heterologous sequence may be inserted into an intergenic region of the poxviral genome (see WO 03/097845). Methods how to insert heterologous sequences into the poxviral genome are known to a person skilled in the art. By way of example the expression cassettes may be inserted into one ore more of the intergenic regions IGR 07/08, IGR 14L/I5L and IGR 136/137 of the MVA genome, in particular the genome of MVA-BN and its derivatives.

According to a preferred embodiment of the present invention the recombinant MVA is MVA-BN or a derivative thereof and the following expression cassettes are inserted into the following insertion sites: (i) an expression cassette expressing Vif-Vpu-Vpr-Rev as fusion protein in this or a different order, wherein Vif, Vpu, Vpr and Rev stand for full length proteins, or parts or derivatives of the full length proteins (see definition above) is inserted into the intergenic region IGR 07/08; (ii) a second expression cassette expressing Nef or a part or derivative thereof, in particular a Nef protein in which N-terminal amino acids are deleted, such as the first 19 amino acids is inserted into IGR 14L/I5L, (iii) a third expression cassette that expresses Tat or a part or derivative thereof, in particular a transdominant Tat and a fourth expression cassette that express a Gag-Pol fusion protein, wherein Gag and Pol stand for full length proteins, or parts or derivatives of the full length proteins are inserted into IGR 136/137. In this example the third and the fourth expression cassette are inserted into the same integration site, wherein the two expression cassettes may be arranged in both possible orders. With respect to the numbering of IGR reference is made to WO 03/097845. It is to be taken into account that IGR 14L/I5L on the one side and IGR 136/137, IGR 07/08 on the other side belong to two different numbering systems which are explained in WO 03/097845.

Most preferrably, the recombinant MVA of the present invention is a recombinant MVA comprising in the viral genome
(i) an expression cassette expressing Vif-Vpu-Vpr-Rev as fusion protein inserted into the intergenic region IGR 07/08,
(ii) a second expression cassette expressing a Nef protein in which the first 19 N-terminal amino acids are deleted inserted into IGR 14L/I5L,
(iii) a third expression cassette expressing a transdominant Tat and a fourth expression cassette expressing a Gag-Pol fusion protein, which are inserted into IGR 136/137.

The recombinant virus according to the present invention may induce a protective immune response: The term “protective immune response” means that the vaccinated subject is able to control in some way an infection with the pathogenic agent against which the vaccination was done. Usually, the animal having developed a “protective immune response” develops milder clinical symptoms than an unvaccinated subject and/or the progression of the disease is slowed down.

The present invention further relates to a pharmaceutical composition or vaccine comprising a recombinant MVA as defined above and, optionally, a pharmaceutically acceptable carrier, diluent, adjuvant and/or additive.

Numerous ways to prepare poxvirus formulations are known to the skilled artisan as well as modes of storage. In this context reference is made to WO 03053463.

Non-limiting examples of auxiliary substances are water, saline, glycerol, ethanol, wetting or emulsifying agents, pH buffering substances, preservatives, stabilizers, or the like. Suitable carriers are typically selected from the group comprising large, slowly metabolized molecules such as, for example, proteins, polysaccharides, polylactic acids, polyglycolitic acids, polymeric amino acids, amino acid copolymers, lipid aggregates, or the like.

For the preparation of vaccines, the recombinant MVA virus according to the invention is converted into a physiologically acceptable form. This can be done based on the experience in the preparation of poxvirus vaccines used for vaccination against smallpox (as described by Stickl, H. et al. Dtsch. med. Wschr. 99, 2386-2392 [1974]). For example, the purified virus is stored at −80° C. with a titer of 5×10⁸ TCID50/m1 formulated in 10 mM Tris, 140 mM NaCl pH 7.4.

In one embodiment, the MVA virus according to the invention is used for the preparation of vaccine shots. For example, about 10² to about 10⁸ particles of the virus are lyophilized in 100 ml of phosphate-buffered saline (PBS) in the presence of 2% peptone and 1% human albumin in an ampoule, preferably a glass ampoule. In another non-limiting example, the vaccine shots are produced by stepwise freeze-drying of the virus in a formulation. In certain embodiments, this formulation can contain additional additives such as mannitol, dextran, sugar, glycine, lactose or polyvinylpyrrolidone or other aids, such as antioxidants or inert gas, stabilizers or recombinant proteins (for example, human serum albumin) suitable for in vivo administration. The glass ampoule is then sealed and can be stored between 4° C. and room temperature for several months. However, as long as no immediate need exists, the ampoule is stored preferably at temperatures below −20° C.

For vaccination or therapy, the lyophilisate may be dissolved in 0.1 to 0.5 ml of an aqueous solution, preferably physiological saline or Tris buffer, and administered either systemically or locally, i.e. parenterally, subcutaneously, intramuscularly, by scarification or any other path of administration know to the skilled practitioner. The mode of administration, the dose and the number of administrations can be optimized by those skilled in the art in a known manner. However, most commonly, a patient is vaccinated with a second shot about one month to six weeks after the first vaccination shot. A third and subsequent shots can be given, preferably 4-12 weeks after the previous shot.

It was surprisingly found that with the recombinant MVA of the present invention a strong antigen specific T-cell response can be induced against more than one of the recombinantly expressed HIV proteins. As already indicated above, this is an unexpected result in view of the observed phenomenon called immunodominance whereby the host immune system responds to only a few of the many possible epitopes in a given immunogen. For MVA vectors previous studies have shown that the immunodominance of non-recombinant vector epitopes prevent induction of a strong CD8 T cell response against a recombinant antigen. (Smith et al., Immunodominance of poxviral-specific CTL in a human trial of recombinant-modified vaccinia Ankara. J. Immunol. 175:8431-8437, 2005.). It was demonstrated that the vaccine-driven T-cell response is predominantly directed against poxviral epitopes. By contrast, use of the recombinant MVA of the present invention resulted in a strong and broad T-cell response to multiple recombinant HIV proteins. In addition, the immune responses were still higher than baseline 20 weeks after receiving the first immunization for all HIV proteins which induced responses. This is a further unexpected result in view of potential pre-existing immunity against the backbone viral vector due to an earlier vaccination against smallpox and the development of neutralizing antibodies.

Accordingly, the present invention further relates to the recombinant MVA as defined above or a pharmaceutical composition or vaccine comprising the recombinant MVA as defined above for inducing a T-cell response to at least three, preferably to at least four, at least five or six HIV proteins in a human patient, wherein the proteins are selected from HIV Gag, Pol, Vpr, Vpu, Rev, and Nef.

The invention also relates to the use of the recombinant MVA as defined above or a pharmaceutical composition or vaccine comprising the recombinant MVA as defined above for the preparation of a medicament for inducing a T-cell response to one or more HIV proteins, especially against three, four, five, six or more HIV proteins, preferably to at least three, at least four, at least five or at least six HIV proteins in a human patient, wherein the proteins are selected from HIV Gag, Pol, Vpr, Vpu, Rev, and Nef.

Likewise, the present invention also relates to a method for inducing a T-cell response to at least three of the HIV proteins selected from Gag, Pol, Vpr, Vpu, Rev and Nef in a human subject comprising administering a recombinant Modified Vaccinia virus Ankara (MVA) as defined above.

The recombinant MVA that is to be administered is preferably an effective amount so that it induces the desired effect, i.e., a T-cell response to at least three, preferably at least four, more preferably at least five, even more preferably at least six of the HIV proteins selected from Gag, Pol, Vpr, Vpu, Rev and Nef in a human subject.

In a particular preferred embodiment the present invention relates to the recombinant recombinant MVA as defined above, wherein the MVA induces a T-cell response in the human subject to at least four of the HIV-1 proteins selected from Gag, Pol, Vpr, Vpu, Rev and Nef.

In a more particular preferred embodiment the present invention relates to the recombinant MVA as defined above, wherein the MVA induces a T-cell response in the human subject to at least five of the HIV-1 proteins selected from Gag, Pol, Vpr, Vpu, Rev and Nef.

In an even more particular preferred embodiment the present invention relates to the recombinant MVA as defined above, wherein the MVA induces a T-cell response in the human subject to at least six of the HIV-1 proteins selected from Gag, Pol, Vpr, Vpu, Rev and Nef.

In a preferred embodiment the HIV-1 proteins to which MVA as defined herein induces a T-cell response to proteins that are selected from Gag, Pol, Vpr, Vpu, Rev and Nef include Gag, Pol and Nef.

In a preferred embodiment said at least three HIV proteins are Gag, Pol and Nef. In another preferred embodiment one of said three HIV proteins is one selected from the group consisting of Gag, Pol, Nef, truncated Nef, Vpr, Vpu, and Ref.

The present invention further relates to the recombinant MVA as defined above or a pharmaceutical composition or vaccine comprising the recombinant MVA as defined above for the treatment and/or prevention of a HIV infection and/or AIDS.

The invention also relates to the use of the recombinant MVA as defined above or a pharmaceutical composition or vaccine comprising the recombinant MVA as defined above for the preparation of a medicament for treatment and/or prevention of a HIV infection and/or AIDS.

It is pointed out that the term “prevention of an HIV infection and/or AIDS” does not mean that the recombinant MVA prevents a HIV infection and/or AIDS in all subjects under all conditions. To the contrary this term covers any statistically significant protective effect even if this effect is rather low.

In a further preferred embodiment the recombinant MVA is administered in a dose of 10⁵ to 5×10⁸ TCID₅₀/ml, preferably in a dose of 2×10⁸ TCID₅₀/ml.

In another preferred embodiment the recombinant MVA is administered intravenously, intramuscularly or subcutaneously.

### BRIEF DESCRIPTION OF THE FIGURES

FIG. 1: MVA-BN®-MAG construct (MVA-mBN120B) expressing the eight HIV proteins Gag, Pol, Tat, Vif, Vpu, Vpr, Rev and Nef.

FIG. 2: HIV-specific T cell responder rates. Percentage of responders calculated from n (number of subjects who were responders) and based on a group size of N=15. RT=reverse transcriptase, CTL=cytotoxic T lymphocytes, HTL=helper T lymphocytes.

FIG. 3 A-D: Median SFU/1×10⁶ PBMC for the indicated HIV-1 proteins. Arrows indicate vaccinations.

### EXAMPLES

The following examples will further illustrate the present invention. It will be well understood by a person skilled in the art that the provided examples in no way may be interpreted in a way that limits the applicability of the technology provided by the present invention to this examples.

### Example 1

Generation of a Recombinant MVA-BN Comprising in the Viral Genome a Truncated nef Gene, a Gag-Pol Fusion Gene, a Transdominant Tat Gene and a Vif-Vpr-Vpu-Rev Fusion Gene, each Under the Control of the ATI Promoter

An MVA vector, mBN87, was generated as described in U.S. Pat. No. 7,501,127, which is hereby incorporated by reference. Briefly, the gag-pol fused gene was obtained by PCR from DNA from HXB2 infected cells. The nef gene was amplified by PCR from DNA of MVA-nef (LAI) to obtain a truncated version. The first 19 aa were deleted resulting in Nef-truncated. The vif and vpu genes were generated by RT-PCR from HIV RNA from a primary isolate MvP-899, while the vpr, rev and tat genes were synthesized by oligo annealing based on the sequence of HXB2. The protein Tat-mutated was created by introducing two mutations in Tat, which are not localized in important epitopes but lead to the loss of transactivating activity. The mutations are the following substitutions: 22 (Cys>Gly) and 37 (Cys>Ser). The DNA constructs were cloned into recombinant vectors.

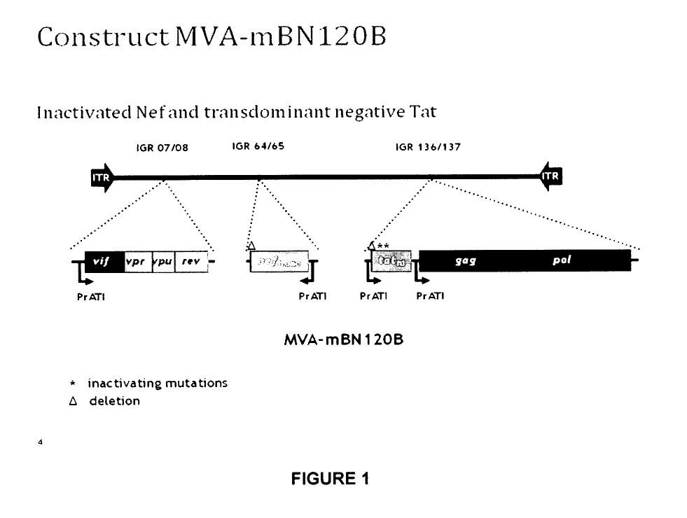

After 5 rounds of plaque purification, the insertion of the foreign DNA (truncated nef gene, a gag-pol fusion gene, a transdominant tat gene, and a vif-vpr-vpu-rev fusion gene) and absence of wild-type virus was confirmed by PCR. The resulting recombinant virus clone was named mBN87A. After 5 plaque-purifications under non selective conditions the recombinant virus MVA-mBN87 B devoid of the selection cassette could be isolated. The identity of the recombinant vector was confirmed by standard methods. In MVA-mBN87B, the vif-vpr-vpu-rev gene doesn't have a stop codon at the end which results in the addition of 31 non-specific amino acids. Thus, a stop codon was added to the fusion gene and by cloning of the new recombinant virus MVA-mBN120B. This construct (see also FIG. 1) was used in preclinical studies in mice and clinical studies in humans.

### Example 2

### Preclinical Studies in Mice

Whether MVA-mBN120B is able to mount a HIV-specific cellular immune response in adult non-transgenic mice (BALB/c) was investigated. The most promising epitopes were selected and for each protein two CD4 and two CD8 T cell peptides were synthesized. On Days 0 and 21, mice were administered subcutaneously (s.c.) with 500 μl of either TBS (Group 1) as reference item or approximately 4×10⁸ TCID₅₀ MVA-mBN120B (Group 2). On Day 35, blood samples were collected from all animals by retro-orbital puncture and processed to serum for potential future analysis. Following blood sampling, the animals were sacrificed by cervical dislocation and spleens necropsied for subsequent analysis of the cellular immune responses by restimulation of splenocytes with the HIV specific peptides encoded in the vaccine inserts using an IFNγ-ELISpot assay. The HIV-protein specific cellular immune responses were determined by restimulation of splenocytes with specific peptides and subsequent detection of IFNγ release from the splenocytes by ELISpot assay. The peptides are as follows, showing peptide denomination, T cell restriction, and peptide sequence:

| | | |
|---|---|---|
| Nef-1 CD4 | FHHVARELHPEYFKNC | (SEQ ID NO: 1) |
| Nef-2 CD4 | DPEREVLEWRFDSRLA | (SEQ ID NO: 2) |
| Nef-3 CD8 | HTQGYFDP | (SEQ ID NO: 3) |
| Nef-4 CD8 | RYPLTFGWC | (SEQ ID NO: 4) |
| Gag-1 CD4 | IYKRWIILGLNK | (SEQ ID NO: 5) |
| Gag-2 CD4 | GLNKIVRMYSPT | (SEQ ID NO: 6) |
| Gag-3 CD8 | AMQMLKETI | (SEQ ID NO: 7) |
| Gag-4 CD8 | EIYKRWIIL | (SEQ ID NO: 8) |
| Pol-1 CD4 | VQNANPDCK | (SEQ ID NO: 9) |
| Pol-2 CD4 | TIKIGGQLK | (SEQ ID NO: 10) |
| Pol-3 CD8 | IFQSSMTKI | (SEQ ID NO: 11) |
| Pol-4 CD8 | QPDKSESEL | (SEQ ID NO: 12) |
| Tat-1 CD4 | FITKALGISYGRK | (SEQ ID NO: 13) |
| Tat-2 CD4 | RQRRRAHQN | (SEQ ID NO: 14) |
| Tat-3 CD8 | QPKTAGTNC | (SEQ ID NO: 15) |
| Tat-4 CD8 | SFITKALGI | (SEQ ID NO: 16) |
| Vif-1 CD4 | KKAKGWMYK | (SEQ ID NO: 17) |
| Vif-2 CD4 | RCEYQAGHN | (SEQ ID NO: 18) |
| Vif-3 CD8 | QYLALAALI | (SEQ ID NO: 19) |
| Vif-4 CD8 | AGHNKVGSL | (SEQ ID NO: 20) |
| Vpu-1 CD4 | KPQKTKGHR | (SEQ ID NO: 21) |
| Vpu-2 CD4 | WAGVEAIIR | (SEQ ID NO: 22) |
| Vpu-3 CD8 | TYGDTWAGV | (SEQ ID NO: 23) |
| Vpu-4 CD8 | AGVEAIIRI | (SEQ ID NO: 24) |
| Vpr-1 CD4 | IVLIEYRKI | (SEQ ID NO: 25) |
| Vpr-2 CD4 | EEALAALVD | (SEQ ID NO: 26) |
| Vpr-3 CD8 | TQPIPIVAI | (SEQ ID NO: 27) |
| Vpr-4 CD8 | VLIEYRKIL | (SEQ ID NO: 28) |
| Rev-1 CD4 | RQARRNRRR | (SEQ ID NO: 29) |
| Rev-2 CD4 | SPQILVESP | (SEQ ID NO: 30) |
| Rev-3 CD8 | SGDSDEELI | (SEQ ID NO: 31) |
| Rev-4 CD8 | LPPLERLTL | (SEQ ID NO: 32) |

Briefly, aliquots containing 500 μg of each peptide were first dissolved in a small volume of dimethyl sulfoxide followed by further dilution with RPMI medium to obtain a stock solution of 1mg/ml (volumes between 5 and 25 μl of acetic acid was additionally required to ensure complete reconstitution of peptides #4, 14, 20, and 21; volumes between 5 and 25 μl were additionally required to ensure complete reconstitution of peptides #20, 22, 23 and 24).

Spleen homogenisation was performed in Dispomix tubes using the “Saw 03” program. Following homogenisation, cell suspensions were transferred into 50 ml tubes, centrifuged, and the erythrocytes were lysed for 5 minutes with red blood cell lysis buffer. Following two washing steps, a small aliquot of the cell suspension was mixed with trypan blue and the cell concentration was calculated by manual counting with a counting chamber (from Madaus). The cell density was adjusted for the individual splenocyte suspensions. Following plating the cells into the ELISpot plate (pre-coated with anti-IFNγ antibody), the peptides were added at a final concentration of 2.5 μg/ml. Duplicate incubations of 2.5×10⁵ splenocytes per well were performed on the horizontally oriented ELISpot plates with splenocytes from different mice being plated horizontally and with different stimuli being plated vertically (i.e. plates 1+5, 2+6, 3+7, 4+8 covered stimulation with peptides #1-8, 9-16, 17-24, 25-32, respectively). On plates 5 and 10, incubations with final concentrations of 0.5 μg Concanavalin A (ConA) and 0.5 μg/ml staphylococcus enterotoxin B (SEB) as positive controls, or with medium control as negative control was performed in row number B, E, or H, respectively. Following an overnight incubation of 19 h, the ELISpot plates were developed as recommended by the supplier.

TABLE 1

| | | Group 1 | | | Group 2 | | |
|---|---|---|---|---|---|---|---|
| | | TBS | | | MVA-mBN120B | | |
| | Peptide | Group | | | Group | | |
| Stimulation | Designation | mean | SEM | N | mean | SEM | N |
| Peptide # 1 | Nef-CD4-1 | 7.6 | 1.9 | 5 | 21.6 | 6.7 | 5 |
| Peptide # 2 | Nef-CD4-2 | 8.8 | 0.8 | 5 | 49.6 | 13.0 | 5 |
| Peptide # 3 | Nef-CD8-1 | 8.0 | 2.4 | 5 | 20.8 | 7.0 | 5 |
| Peptide # 4 | Nef-CD8-2 | 8.8 | 2.1 | 5 | 27.2 | 11.1 | 5 |
| Peptide # 5 | Gag-CD4-1 | 8.8 | 3.0 | 5 | 30.8 | 4.8 | 5 |
| Peptide # 6 | Gag-CD4-2 | 9.6 | 3.5 | 5 | 28.4 | 7.6 | 5 |
| Peptide # 7 | Gag-CD8-1 | 10.0 | 2.7 | 5 | 304.8 | 84.2 | 5 |
| Peptide # 8 | Gag-CD8-2 | 6.0 | 2.5 | 5 | 158.0 | 47.3 | 5 |
| Peptide # 9 | Pol-CD4-1 | 6.8 | 1.4 | 5 | 22.0 | 7.3 | 5 |
| Peptide # 10 | Pol-CD4-2 | 8.4 | 1.9 | 5 | 20.4 | 5.4 | 5 |
| Peptide # 11 | Pol-CD8-1 | 9.6 | 1.7 | 5 | 26.8 | 11.0 | 5 |
| Peptide # 12 | Pol-CD8-2 | 7.6 | 2.6 | 5 | 20.8 | 6.1 | 5 |
| Peptide # 13 | Tat-CD4-1 | 8.4 | 2.6 | 5 | 20.8 | 2.7 | 5 |
| Peptide # 14 | Tat-CD4-2 | 10.4 | 2.3 | 5 | 20.8 | 2.1 | 5 |
| Peptide # 15 | Tat-CD8-1 | 6.0 | 1.4 | 5 | 19.6 | 2.5 | 5 |
| Peptide # 16 | Tat-CD8-2 | 8.8 | 2.9 | 5 | 16.0 | 7.3 | 5 |
| Peptide # 17 | Vif-CD4-1 | 8.0 | 3.0 | 5 | 21.2 | 5.3 | 5 |
| Peptide # 18 | Vif-CD4-2 | 9.2 | 2.2 | 5 | 31.2 | 6.5 | 5 |
| Peptide # 19 | Vif-CD8-1 | 7.2 | 1.6 | 5 | 27.6 | 6.0 | 5 |
| Peptide # 20 | Vif-CD8-2 | 6.4 | 3.5 | 5 | 25.2 | 8.5 | 5 |
| Peptide # 21 | Vpu-CD4-1 | 4.8 | 1.7 | 5 | 22.4 | 6.0 | 5 |
| Peptide # 22 | Vpu-CD4-2 | 7.2 | 2.1 | 5 | 17.2 | 3.2 | 5 |
| Peptide # 23 | Vpu-CD8-1 | 11.2 | 2.4 | 5 | 21.6 | 3.3 | 5 |
| Peptide # 24 | Vpu-CD8-2 | 7.2 | 1.9 | 5 | 24.0 | 7.3 | 5 |
| Peptide # 25 | Vpr-CD4-1 | 6.8 | 2.0 | 5 | 20.0 | 6.9 | 5 |
| Peptide # 26 | Vpr-CD4-2 | 6.0 | 2.6 | 5 | 18.8 | 4.5 | 5 |
| Peptide # 27 | Vpr-CD8-1 | 8.8 | 2.1 | 5 | 24.0 | 4.3 | 5 |
| Peptide # 28 | Vpr-CD8-2 | 6.4 | 3.1 | 5 | 24.8 | 6.6 | 5 |
| Peptide # 29 | Rev-CD4-1 | 7.6 | 2.6 | 5 | 20.8 | 6.5 | 5 |
| Peptide # 30 | Rev-CD4-2 | 8.0 | 2.8 | 5 | 27.2 | 6.4 | 5 |
| Peptide # 31 | Rev-CD8-1 | 8.8 | 3.0 | 5 | 20.4 | 6.4 | 5 |
| Peptide # 32 | Rev-CD8-2 | 7.2 | 1.6 | 5 | 19.6 | 4.6 | 5 |
| Con A | n.a. | 443.6 | 30.0 | 5 | 212.0* | 59.8 | 5 |
| SEB | n.a. | 247.6 | 38.7 | 5 | 154.8* | 56.1 | 5 |
| Medium | n.a. | 6.8 | 2.6 | 5 | 20.8 | 7.0 | 5 |

n.a. = not applicable

Three peptides were identified to be able to mount a HIV-specific cellular response. From these peptides, the highest IFN1 responses were determined following stimulation with the H2-Kd restricted CD8 T cell specific peptide “Gag-CD8-1”. This is not surprising, since this peptide is frequently cited in the literature (e.g. Liu et al., Vaccine, 2006, 24, page 3332). The second Gag-specific CD8 T cell restricted peptide “Gag-CD8-2” was also able to induce a good specific IFN1 release in all mice. This peptide was so far only described by Shinoda et al. (Vaccine, 2004, 22, page 3676) to induce a specific CTL response in the context of a longer peptide. However, the longer peptide described in the literature not only contains the CD8 T cell epitope, but also additional predictable (and therefore potential) CD8 but also CD4 T cell epitopes. Thus, it is shown for the first time that “Gag-CD8-2” is able to induce a specific IFN1 release. Based on epitope prediction, “Gag-CD8-2” is an H2-Dd restricted CD8 T cell epitope. The third responsive peptide “Nef-CD4-2” was able to induce a specific IFN1 release in the majority of BALB/c mice. This peptide was already described by Mitchel et al. (AIDS Research and Human Retroviruses, 1992, 8, page 469) as a peptide to which a proliferative response and a cytolytic activity could be determined. From the epitope prediction, this peptide was identified as being primarily restricted to CD4 T cells (scores of 9.6 for the I-Ed molecule and 9.1 for the IAd molecule were identified in the PredBALB/C data base, whereas scores for the CD8 T cell restricted H2d molecules were below 7.9). Surprisingly, peptides other than the three responsive ones, e.g. “Nef-CD4-1” or “Tat-CD8-1”, which had been selected based on published literature results were not found to be able to induce a specific IFN1 release. The reason for this discrepancy is not known.

In summary, the immunogenicity study in BALB/c mice with MVA-mBN120B demonstrated not only that the HIV-Multiantigen MVA-construct is immunogenic, but revealed also that the immune response is directed against at least 2 proteins encoded in the recombinant MVA product (i.e. Nef and Gag specific responses were detected), that the CD8 T cell restricted immune responses are not limited to a single CD8 T cell molecule (since both H2-Kd and H2-Dd responses are induced), and that both CD8 and also CD4 T cell restricted responses were induced by the MVA-construct. Furthermore, these results indicate that, at least, the Nef-gene and the Gag-gene are expressed from the vector in vivo.

### Example 3

### Clinical Studies in Humans

In a Phase I study safety, reactogenicity and immunogenicity of a recombinant MVA-BN® vaccine expressing 8 out of 9 genes from HIV-1 clade B subgroup, (including a gag-pol fusion, vpr, vpu, vif, rev, tat, and nef) was evaluated in 15 HIV-1 infected subjects. This safety testing encompassed an analysis of solicited and unsolicited local and systemic adverse reactions. Furthermore, cellular and humoral immune responses to the vector were assessed. The collected specimens were also used to develop assays to specifically analyze the HIV-specific immune responses induced by the study vaccine MVA-mBN120B in order to establish the potential of such a homologous prime-boost vaccine approach to induce a broad cell-mediated response to different HIV antigens. In this Phase I trial, 15 HIV-1 infected patients stable on HAART (Highly Active Anti-Retroviral Therapy) with CD4 counts>350/μl received three vaccinations with 2×10⁸ MVA-BN®-MAG at Weeks 0, 4, and 12. Solicited Adverse events (AE) were documented on diary cards, unsolicited AEs and cardiac signs and symptoms were captured throughout the study until the follow up visit at Week 20. Disease specific parameters such as plasma HI-viral load and CD4 counts of the patients were determined. Vaccinia specific humoral immune responses were measured by ELISA; cellular immune responses to the HIV-1 inserts as well as to vaccinia were assessed by an Interferon-γ (IFN-γ) ELISPOT assay using 15-mer peptides with an 11 amino acids overlap as a stimulant (for inducing HIV responses) and MVA-BN® at an multiplicity of infection (MOI) of 1 (for inducing vaccinia responses) respectively in a batched analysis.

The study was a mono-centric, open-label, Phase I study conducted to assess safety and reactogenicity of the recombinant MVA HIV multiantigen vaccine in HIV-infected subjects with CD4 counts>350 cells/μl.

Subjects received immunizations at Day 0 and after 4 and 12 weeks with a dose of 2×10⁸ tissue culture infectious dose 50 (TCID₅₀) MVA-mBN120B. The vaccine was administered subcutaneously.

The study consisted of a screening period of up to three weeks and an active study period (a 12-week priming phase and an 8-week boosting phase) of up to 20 weeks. The total duration of the study per subject was up to 23 weeks.

Eligible subjects entered the active study phase starting with Visit 1. At Visit 1, all subjects received the first MVA-BN120B vaccination, administered subcutaneously. All subjects received a second vaccination four weeks later at Visit 3 and a third vaccination 12 weeks later (after Visit 1) at Visit 5. Each immunization consisted of two administrations of MVA-mBN120B each with a dose of 1×10⁸ TCID₅₀ per administration.

The vaccine was administered subcutaneously by injecting 0.5 ml of MVA-mBN120B in the deltoid region of each arm. Subjects received three immunizations: one at Week 0, one after four weeks and one after 12 weeks. Any adverse event (AE) that occurred during or after the vaccination was recorded.

The following procedures and investigations were performed at the respective scheduled visit. The Enzyme Linked Immunospot (ELISPOT) assay used for the quantitative in vitro determination of Interferon-gamma (IFN-γ) producing cells in cryopreserved Peripheral Blood Mononuclear Cells (PBMC) was performed after stimulation with live Vaccinia virus (VV): Modified Vaccinia Virus Ankara-Bavarian Nordic (MVA-BN®) or Vaccinia Virus Western Reserve (W-WR).

Briefly, 96 well filter plates (HTS plates, Millipore) were coated with a capture antibody (against IFN-γ according to the manufacturer's instructions (BD Biosciences, IFN-γ ELISPOT pair) at 4° C. overnight. Subsequently resuscitated PBMC were added to the wells in a concentration of 200,000 cells/200 μl final volume in combination with MVA-BN® at an MOI of 1. Following an incubation period (overnight at 37° C./5% CO₂) the wells were washed and a biotin-labelled detection antibody in PBS/9% FCS was added. After washing with PBS/0.05% Tween 20, streptavidin-coupled horse radish peroxidase (HRP, BD Biosciences) was added to the wells, followed by a washing step and the addition of a precipitating substrate (AEC, 3-Amino-9-Ethylcarbazole, BD Biosciences). The number of cytokine producing cells was determined by counting the spots using a CTL S5 Microanalyzer. Reported values are background corrected and normalized to 1×10⁶ PBMC.

To assess the vaccinia specific cellular response, cells were stimulated with live MVA-BN® at an MOI of 1.

To assess the HIV specific cellular response, cells were stimulated with peptide pools (15-mers with 11 aa overlap) at a final concentration of 5 μg/ml per peptide.

Fifteen peptide pools were used for stimulation and are depicted in Table 2.

TABLE 2

| | | | Number of |
|---|---|---|---|
| protein | Pool # | Peptides | Peptides |
| p17 (Gag) | 1 | 100% coverage | 31 |
| p24 (Gag) | 2 | N terminal part, 100% | 28 |
| | | coverage | |
| | 3 | C terminal part, 100% | 27 |
| | | coverage | |
| Protease (Pol) | 4 | Immunogenic regions only | 11 |
| RT (Pol) | 5 | Immunogenic regions only | 22 |
| Integrase (Pol) | 6 | Immunogenic regions only | 10 |
| Nef | 7 | Predicted poorly | 14 |
| | | immunogenic | |
| | 8 | Predicted poorly | 11 |
| | | immunogenic | |
| | 9 | Predicted highly immunogenic | 10 |
| | 10 | Predicted highly immunogenic | 9 |
| Tat | 11 | 100% coverage | 10 |
| Vif | 12 | Immunogenic regions only | 16 |
| p2p7 | 13 | 100% coverage | 11 + 8 + 6 = |
| Vpr | | Immunogenic regions only | 25 |
| Rev | | Immunogenic regions only | |
| POLYTOPE | 14 | | 15 |
| HTL | | | |
| POLYTOPE | 15 | A2, A3, B7 restricted | 13 |
| CTL | | | |

Peptides were synthesized at more than 90% purity as confirmed by high-performance liquid chromatography (Metabion, Martinsried, Germany and Proimmune, UK).

A vector or HIV-MAG-specific signal was defined by a frequency of at least 50 SFU per 1×10⁶ cells after correction for background (subtraction of SFU/1×10⁶ non-stimulated cells/≧two-fold above background). The number of SFU/1×10⁶ cells after correction for background was reported.

Vector or HIV-MAG-specific T cell responses were defined as either the occurrence of a signal in a subject who had no signal at Baseline, or a relative increase by a factor of at least 1.7 over the Baseline value in subjects who had a signal at Baseline.

Subjects who had responses at one or more post-Baseline visits were classified as responders.

A specific signal was defined (for each subject, visit and stimulation condition) by subtracting the numbers of spot-forming cells in background (non-stimulated) wells from those appearing in corresponding experimental (stimulated) wells. Specific signals of less than 50 spot forming units (SFU) were returned to zero for the calculation of responses.

A positive specific response was defined when either there was the appearance of a positive specific signal equal to or above the assay cut-off of 50 SFU per 1×10⁶ PBMC in subjects who were previously below the assay cut off at baseline (V1); or a rise of a factor of at least 1.7 in the number of SFU from the baseline (V1) signal for subjects who had a baseline (V1) signal equal to or above the assay cut-off value. Otherwise the response was defined as negative, except in the case that either the respective post-baseline or the baseline values were missing; then the response status was defined as missing.

Subjects could have more than one response over the multiple post-baseline visits but only one response was required to be considered a specific responder.

HIV peptide stimulation was analyzed at three levels for all subjects and a separate analysis for responders only, by stimulating pool (1-15), by protein/polyprotein (gag, pol, nef, tat, vif, mixed), and by vaccine (i.e. including all HIV proteins).

Descriptive statistics were derived by stimulation condition (including stimulation with HIV-MAG peptides and live MVA-BN®) for all sampling points and included the number of observations, arithmetic mean and standard deviation (SD), median and range of the number of SFU. This was performed for all subjects and a separate analysis was performed for responders only at all three levels of analysis (i.e. for responder on the pool level, for responder on the protein/polyprotein and responder on the vaccine level). The number and percentage of positive specific responders (responder rate) along with the 95% Clopper-Pearson confidence interval was tabulated for each pool, each protein/polyprotein and for the overall HIV-MAG vaccine as well as for MVA-BN®. The percentage was calculated based on the number of subjects included in the specific analysis. A subject only needed to respond to one pool at the protein/polyprotein and vaccine level to be considered a responder. The same was true for vector-specific responder rates which were tested using only one stimulating condition (stimulation by MVA-BN®).

The breadth of the HIV specific response was represented by a cumulative depiction of subject protein/polyprotein responses using the following categories: number of subjects with a response to 1 or more, 2 or more, 3 or more, 4 or more 5 or more, and 6 proteins/polyproteins.

Prior to vaccination as determined by ELISPOT, 87% of the subjects generated cellular immune responses to gag, 60% to nef, 53% to CTL epitopes, 40% to mixed proteins and HTL epitopes, 33% to pol and only 7% to tat and vif.

Responder rates for each peptide pool, protein/polyprotein and HIV vaccine (HIV-MAG) are summarized in Table 3 and also reveal the time points at which responses were detected. The use of single responses to define a responder was defined in the SAP and is a higher sensitivity method for examining responses; however, this method may be prone to higher false positive rates. For this reason, a supplementary analysis has been performed using a higher stringency definition of responder; two responses are required to be defined as a responder.

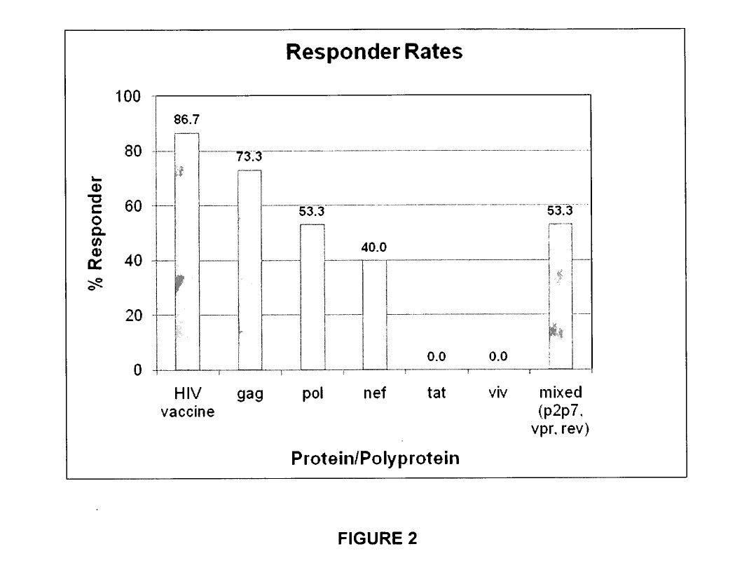

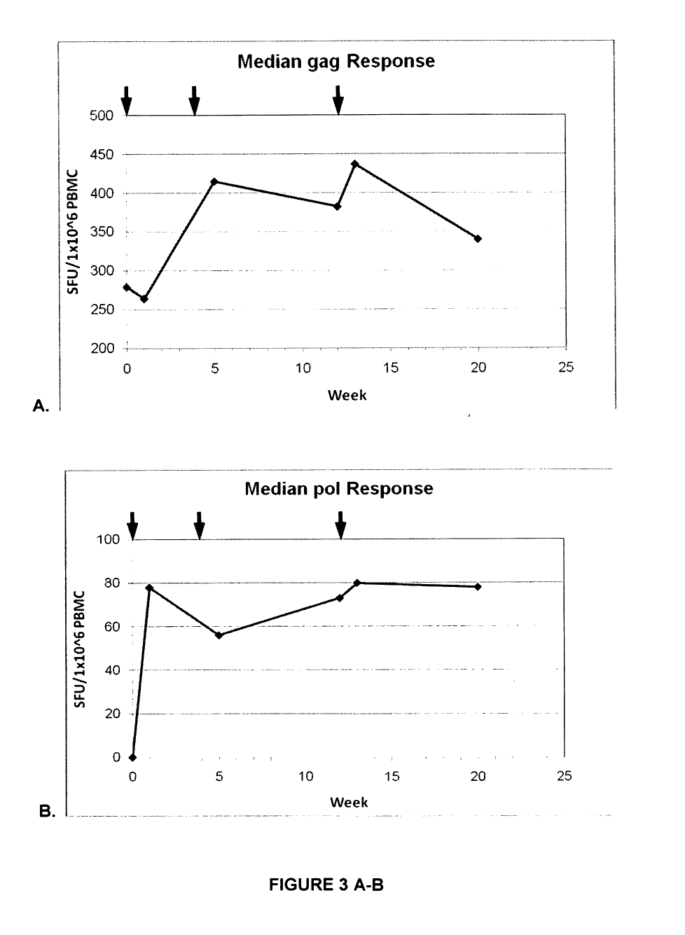

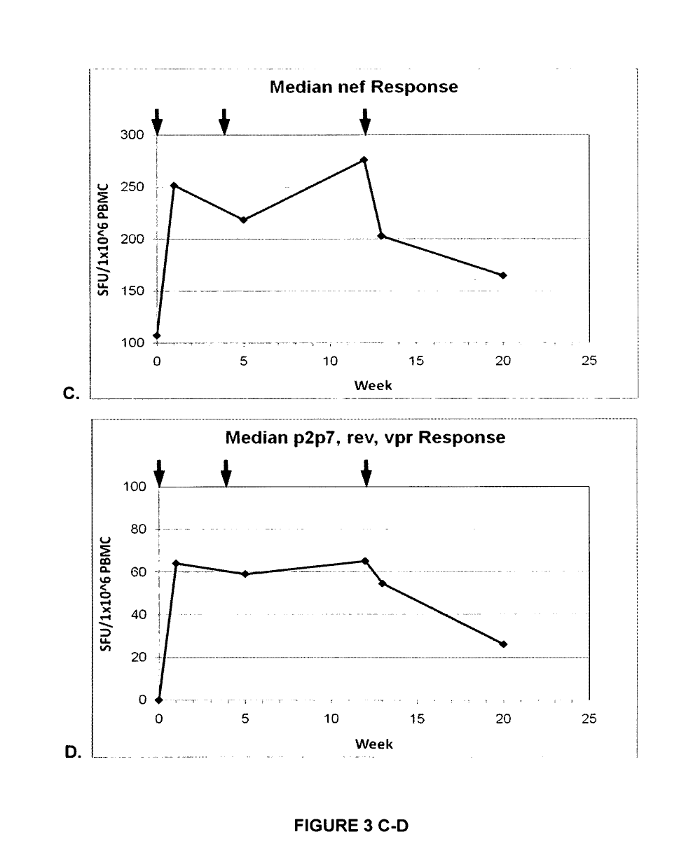

As also shown in FIG. 2, response rates, which imply new responses or increased responses over Baseline values, were high to the HIV proteins coded within the MVA-BN® vaccine-vector with 87% (13/15) of the subjects responding. Even using the higher stringency definition of responder, 80% of the subjects were responders to the HIV components. The highest proportion of subjects responded to gag (73%, 11/15, see FIG. 2). Within gag, p24 resulted in higher responder rates (40% and 47% for the two gag-p24 pools respectively) than did p17 (20%). Even using a higher stringency definition of responder, 60% of the subjects responded to gag. Responder rates to pol and the mixed protein pool were similar (53%, 8/15, see also FIG. 2) and within pol, the protease and RT had the highest and equal responder rates (27%) and were followed by integrase with a 20% responder rate. Since the mixed pool contained peptides from multiple proteins, the most immunogenicpeptides could not be detrminmed. Using the higher stringency definition of responder rate still resulted in high responder rates to both pol (33%) and mixed (40%). Nef responder rate was 40% (6/15, see FIG. 2) with responses to pool 4 being the highest (27%). Nef response was 27% using the higher stringency definition of responder rate. A total of 7 subjects (46.7%) and 1 subject (6.7%) responded to HTL and CTL polytope peptides and no responders were detected for both Tat and Vif.

TABLE 3

| | | | | Pool | | Pool | | Protein | | Protein | |
|---|---|---|---|---|---|---|---|---|---|---|---|
| | | | | Responders | | Responders | | Responders | | Responders | |
| | Protein/ | | | (1 or more | | (2 or more | | (1 or more | | (2 or more | |
| | Poly- | | Responses | responses) | | responses) | | responses) | | responses) | |
| Pool | protein | Subject | (Weeks) | n (%) | | n (%) | | n (%) | | n (%) | |
| 1 | Gag- | 006 | 5, 13 | 3 | (20.0) | 2 | (13.3) | | | | |
| | p17 | 008 | 12 | | | | | | | | |
| | | 013 | 1, 12 | | | | | | | | |
| 2 | Gag- | 001 | 12 | 6 | (40.0) | 4 | (26.7) | | | | |
| | p24- | 002 | 20 | | | | | | | | |
| | NH₃ | 008 | 12, 13, 20 | | | | | | | | |
| | | 010 | 1, 5, 12, 13, 20 | | | | | | | | |
| | | 011 | 1, 5, 12, 13, 20 | | | | | | | | |
| | | 015 | 1, 5 | | | | | | | | |
| 3 | Gag- | 004 | 1, 5, 12, 13 | 7 | (46.7) | 5 | (33.3) | 11 | (73.3) | 9 | (60.0) |
| | p24- | 006 | 13 | | | | | | | | |
| | COOH | 008 | 12, 20 | | | | | | | | |
| | | 009 | 5, 12 | | | | | | | | |
| | | 011 | 5, 13, 20 | | | | | | | | |
| | | 014 | 1, 5, 12, 13, 20 | | | | | | | | |
| | | 015 | 1 | | | | | | | | |
| 4 | Pol- | 004 | 5 | 4 | (26.7) | 3 | (20.0) | | | | |
| | Protease | 006 | 1, 5, 13 | | | | | | | | |
| | | 014 | 12, 13 | | | | | | | | |
| | | 015 | 1, 5 | | | | | | | | |
| 5 | Pol-RT | 001 | 1, 12, 13 | 4 | (26.7) | 2 | (13.3) | | | | |
| | | 006 | 1, 5, 12, 13, 20 | | | | | | | | |
| | | 008 | 12 | | | | | | | | |
| | | 014 | 13 | | | | | | | | |
| 6 | Pol- | 005 | 12 | 3 | (20.0) | 2 | (13.3) | 8 | (53.3) | 5 | (33.3) |
| | Integrase | 006 | 5, 12, 13, 20 | | | | | | | | |
| | | 010 | 1, 13, 20 | | | | | | | | |
| 7 | Nef-1 | 006 | 12 | 1 | (6.7) | 0 | (0.0) | | | | |
| 8 | Nef-2 | 005 | 1, 13 | 2 | (13.3) | 1 | (6.7) | | | | |
| | | 015 | 1 | | | | | | | | |
| 9 | Nef-3 | 015 | 1, 5 | 1 | (6.7) | 1 | (6.7) | | | | |
| 10 | Nef-4 | 001 | 12, 13 | 4 | (26.7) | 3 | (20.0) | 6 | (40.0) | 4 | (26.7) |
| | | 004 | 1 | | | | | | | | |
| | | 013 | 1, 5, 12, 13 | | | | | | | | |
| | | 015 | 1, 5 | | | | | | | | |
| 11 | Tat | None | None | 0 | (0.0) | 0 | (0.0) | 0 | (0.0) | 0 | (0.0) |
| 12 | Vif | None | None | 0 | (0.0) | 0 | (0.0) | 0 | (0.0) | 0 | (0.0) |
| 13 | Mixed | 001 | 5, 12, 13, 20 | 8 | (53.3) | 6 | (40.0) | 8 | (53.3) | 6 | (40.0) |
| | | 004 | 1 | | | | | | | | |
| | | 006 | 1, 12, 13, 20 | | | | | | | | |
| | | 008 | 12, 13, 20 | | | | | | | | |
| | | 009 | 1, 5, 12, 13, 20 | | | | | | | | |
| | | 010 | 1 | | | | | | | | |
| | | 012 | 1, 5 | | | | | | | | |
| | | 013 | 1, 12 | | | | | | | | |
| 14 | HTL | 004 | 1 | 7 | (46.7) | 4 | (26.7) | 7 | (46.7) | 4 | (26.7) |
| | | 006 | 1, 5, 12, 13, 20 | | | | | | | | |
| | | 008 | 1, 12, 20 | | | | | | | | |
| | | 009 | 20 | | | | | | | | |
| | | 012 | 1, 5, 13 | | | | | | | | |
| | | 014 | 5, 13, 20 | | | | | | | | |
| | | 015 | 1 | | | | | | | | |
| 15 | CTL | 006 | 1, 12, 20 | 1 | (6.7) | 1 | (6.7) | 1 | (6.7) | 1 | (6.7) |

| | Responders (1 or more responses) | Responders (2 or more responses) |
|---|---|---|
| | n (%) | n (%) |
| HIV-MAG^{b} | 13 (86.7) | 12 (80.0) |

Table 3: ELISPOT Responder Rates by Visit and Stimulating Condition (Pool, Protein & HIV-MAG; N = 15)
^{a}Response to Protein. A subject was a protein-specific responder if he had at least one positive response for at least one pool for the protein at a post-baseline visit.
^{b}Response to HIV-MAG. A subject was a HIV-MAG-specific responder if he had at least one positive response for at least one HIV protein (gag, pol, nef, tat, vif or mixed [p2p7, rev, vpr]) at a post-baseline visit.
N = number of subjects in the specified group, n = number of subjects who were responders, % = percentage based on N.
Immunizations were given at Week 0, Week 4 and Week 12.

The breadth of HIV-specific T cell response refers to the numbers of proteins/polyproteins for which subjects generated new or increased T cell responses. Table 4 shows the breath of response to HIV proteins. Vaccination resulted in the generation of responses to several proteins in most subjects. Responses to up to four different proteins/polyproteins including gag, pol, tat, vif, nef and mixed (p2p7, vpr, rev) are shown in Table 4. 66.7% of all subjects responded to at least two and 46.7% to at least three proteins/polyproteins.

TABLE 4

N = 15

| Number of proteins/polyproteins | R+ | (%) | 95% CI |
|---|---|---|---|
| At least 1 protein/polyprotein | 13 | (86.7) | 59.5, 98.3 |
| At least 2 proteins/polyproteins | 10 | (66.7) | 38.4, 88.2 |
| At least 3 proteins/polyproteins | 7 | (46.7) | 21.3, 73.4 |
| At least 4 proteins/polyproteins | 3 | (20.0) | 4.3, 48.1 |

N = number of subjects in the specified group,
R+ = number of subjects who were responders to any protein mentioned above),
% = percentage based on N,
95% CI = Clopper-Pearson confidence interval, lower limit and upper limit.

All 15 subjects received 3 vaccinations and were followed up until end of the study. No serious AEs were reported and no study subject was withdrawn due to a related AE. All subjects reported general (mostly nausea) and local reactions (mostly induration) with one grade 3 event (injection site pain). Thus, administration of MVA-BN®-MAG was well tolerated in HIV-1 infected subjects.

All subjects responded to vaccinia. FIG. 4 A-D demonstrates median SFU/1×10⁶ PBMC for the indicated HIV-1 proteins. Arrows indicate vaccinations. The results may be summarized as follows:

Gag responders (11/15 subjects): Median peak of 437 SFU/1×10⁶ PBMC at week 13, one week following the third immunization.

Pol responders (8/15 subjects): Median peak of 80 SFU/1×10⁶ PBMC at week 13, one week following the third immunization.

Nef responders (6/15 subjects): Median peak of 276 SFU/1×10⁶ PBMC at week 12, eight weeks following the second immunization.

Mixed pool (p2p7, vpr, rev) responders (8/15 subjects): Median peak of 65 SFU/1×106 PBMC at week 12, eight weeks following the second immunization.

Gag, pol, nef and mixed responsive IFN-γ secreting PBMCs remained higher than baseline 20 weeks after the first immunization.

Median SFU values for vaccinia-specific responders reached a peak of 350 SFU/1×10⁶ PBMC at Week 12, eight weeks following the second immunization, and was not further increased following the third vaccination. As observed for HIV responses the number of vaccinia responsive IFN-γ secreting PBMCs remained higher than baseline 20 weeks after the first immunization. Anti-vaccinia antibody seroconversion rate reached 100.0% at Week 5 (one week after the second vaccination) and remained at 100% for the duration of the study. ELISA GMT's revealed a slight increase 1 week after the first vaccination and strong booster responses. Vaccinia-specific antibody titers reached a peak of 876 one week after the third immunization and remained much higher than baseline 20 weeks after the first immunization.

The MVA-BN®-MAG HIV vaccine candidate was well tolerated in HIV-1 infected subjects. HIV-specific T cell responder rate was 86.7% and the vaccinia-specific responder rate was 100%. A broad cellular immune response against the four HIV protein/polyprotein pools (gag, pol, nef and mixed [p2p7-vpr-rev]) was observed; 66.7% of all subjects responded to at least two and 46.7% to at least three HIV-1 proteins/polyproteins. Median T cell responses remained higher than baseline 20 weeks after the first immunization for all HIV proteins which induced responses. This was also true for the vaccinia-specific T cell response. Thus, the MVA-BN®-MAG vaccine was able to induce a broad immune response to multiple HIV-1 proteins and to vaccinia and the responses were still higher than baseline 20 weeks after receiving the first immunization.

The present invention also includes the following items:

1. Recombinant Modified Vaccinia virus Ankara (MVA) comprising in the viral genome one or more expression cassettes for the expression of at least three HIV proteins selected from Gag, Pol, Tat, Vif, Vpu, Vpr, Rev and Nef or parts or derivatives of said proteins for use as medicament or vaccine.

2. Recombinant Modified Vaccinia virus Ankara (MVA) according to item 1 comprising in the viral genome one or more expression cassettes for the expression of at least six HIV proteins selected from Gag, Pol, Tat, Vif, Vpu, Vpr, Rev and Nef or parts or derivatives of said proteins.

3. Recombinant MVA according to item 1 or 2, wherein a part of a HIV protein is a protein comprising at least 10 consecutive amino acids of the corresponding full length protein.

4. Recombinant MVA according to any one of the preceding items, wherein a derivative of a HIV protein is an amino acid sequence showing a homology of at least 50% to the respective HIV protein in the HIV-1 isolate HXB2R (genebank accession number K03455)

5. Recombinant MVA according to any one of the preceding items, wherein the MVA genome comprises an expression cassette coding for a fusion protein comprising Vif, Vpu, Vpr and Rev, an expression cassette coding for Nef, an expression cassette coding for a Gag-Pol fusion protein and an expression cassette coding for Tat.

6. Recombinant MVA according to any one of the preceding items, wherein Nef is an N-terminal truncated Nef and/or wherein Tat is a transdominant Tat.

7. Recombinant MVA according to any one of the preceding items comprising in the viral genome
(i) an expression cassette expressing Vif-Vpu-Vpr-Rev as fusion protein inserted into the intergenic region IGR 07/08,
(ii) a second expression cassette expressing a Nef protein in which the first 19 N-terminal amino acids are deleted inserted into IGR 14L/15L,
(iii) a third expression cassette expressing a transdominant Tat and a fourth expression cassette expressing a Gag-Pol fusion protein, which are inserted into IGR 136/137.

8. Pharmaceutical composition or vaccine comprising a recombinant MVA according to any one of items 1 to 7 and, optionally, a pharmaceutically acceptable carrier, diluent, adjuvant and/or additive.

9. Recombinant MVA according to any one of items 1 to 7 or the pharmaceutical composition or vaccine according to item 8 for inducing a T-cell response to at least three HIV proteins in a human patient, wherein the proteins are selected from HIV Gag, Pol, Vpr, Vpu, Rev, and Nef.

10. Use of the recombinant MVA according to any one of items 1 to 7 or the pharmaceutical composition or vaccine according to item 8 for the preparation of a medicament for inducing a T-cell response to at least three HIV proteins in a human patient, wherein the proteins are selected from HIV Gag, Pol, Vpr, Vpu, Rev, and Nef.

11. Recombinant MVA, pharmaceutical composition or vaccine according to item 9 or use according to item 10, wherein the HIV proteins are Gag, Pol and Nef.

12. Recombinant MVA, pharmaceutical composition or vaccine according to item 9 or use according to item 10 for inducing a T-cell response to at least four of the HIV proteins selected from Gag, Pol, Vpr, Vpu, Rev, and Nef.

13. Recombinant MVA, pharmaceutical composition or vaccine according to item 9 or 12 or use according to item 10 or 12 for inducing a T-cell response to the HIV proteins Gag, Pol, Vpr, Vpu, Rev, and Nef.

14. Recombinant MVA according to any one of items 1 to 7 or the pharmaceutical composition or vaccine according to item 8 for the treatment and/or prevention of a HIV infection and/or AIDS.

15. Use of the recombinant MVA according to any one of items 1 to 7 or the pharmaceutical composition or vaccine according to item 8 for the preparation of a medicament for the treatment and/or prevention of a HIV infection and/or AIDS.

1-25. (canceled)

26. A method for inducing a T-cell response to at least four HIV-1 proteins in a human patient comprising administering a dosage of 10⁷ to 10⁹ TCID₅₀ of a modified vaccinia Ankara (MVA) virus vector encoding at least six HIV-1 proteins or an antigenic part thereof comprising at least 10 consecutive amino acids of the protein;
wherein the proteins are selected from HIV-1 Gag, Pol, Tat, Vif, Vpr, Vpu, Rev, and Nef; and
wherein the MVA virus vector induces a T-cell response in the patient to at least four of the HIV-1 proteins.

27. The method of claim 26, wherein the MVA virus vector induces a T-cell response in the patient to at least five of the HIV-1 proteins.

28. The method of claim 26, wherein the HIV-1 proteins are Gag, Pol, Vpr, Vpu, Rev, and Nef.

29. The method of claim 26, wherein one of the proteins is an HIV-1 Gag protein.

30. The method of claim 26, wherein one of the proteins is an HIV-1 Pol protein.

31. The method of claim 26, wherein one of the proteins is an HIV-1 Nef protein.

32. The method of claim 31, wherein the protein is a truncated HIV-1 Nef protein.

33. The method of claim 26, wherein one of the proteins is an HIV-1 Vpr protein.

34. The method of claim 26, wherein one of the proteins is an HIV-1 Vpu protein.

35. The method of claim 26, wherein one of the proteins is an HIV-1 Rev protein.

36. The method of claim 26, wherein the vector comprises a coding sequence for HIV-1 Gag-Pol protein.

37. The method of claim 36, wherein the vector further comprises a coding sequence for a truncated HIV-1 Nef protein.

38. The method of claim 37, wherein the vector further comprises a coding sequence for HIV-1 Vif, Vpr, Vpu, and Rev proteins.

39. The method of claim 38, wherein the vector further comprises a coding sequence for HIV-1 Tat protein.

40. The method of claim 39, wherein a dosage of 10⁸ to 10⁹ TCID₅₀ of the vector is administered to the patient.

41. The method of claim 40, wherein a dosage of 2×10⁸ TCID₅₀ of the vector is administered to the patient.

42. The method of claim 26, wherein the MVA is MVA-BN.

43. The method of claim 26, wherein the patient is infected with HIV-1.

44. The method of claim 26, wherein the MVA virus vector induces a T-cell response in the patient to six of the HIV-1 proteins.

45. The method of claim 26, wherein the MVA virus vector is administered at least three times.

46. The method of claim 45, wherein the MVA virus vector is administered at week 0, 4 and 12.


SEQUENCE LISTING (32 sequences)

SEQ ID NO: 1 - protein, 16 aa, Human immunodeficiency virus
FHHVARELHPEYFKNC

SEQ ID NO: 2 - protein, 16 aa, Human immunodeficiency virus
DPEREVLEWRFDSRLA

SEQ ID NO: 3 - protein, 8 aa, Human immunodeficiency virus
HTQGYFDP

SEQ ID NO: 4 - protein, 9 aa, Human immunodeficiency virus
RYPLTFGWC

SEQ ID NO: 5 - protein, 12 aa, Human immunodeficiency virus
IYKRWIILGLNK

SEQ ID NO: 6 - protein, 12 aa, Human immunodeficiency virus
GLNKIVRMYSPT

SEQ ID NO: 7 - protein, 9 aa, Human immunodeficiency virus
AMQMLKETI

SEQ ID NO: 8 - protein, 9 aa, Human immunodeficiency virus
EIYKRWIIL

SEQ ID NO: 9 - protein, 9 aa, Human immunodeficiency virus
VQNANPDCK

SEQ ID NO: 10 - protein, 9 aa, Human immunodeficiency virus
TIKIGGQLK

SEQ ID NO: 11 - protein, 9 aa, Human immunodeficiency virus
IFQSSMTKI

SEQ ID NO: 12 - protein, 9 aa, Human immunodeficiency virus
QPDKSESEL

SEQ ID NO: 13 - protein, 13 aa, Human immunodeficiency virus
FITKALGISYGRK

SEQ ID NO: 14 - protein, 9 aa, Human immunodeficiency virus
RQRRRAHQN

SEQ ID NO: 15 - protein, 9 aa, Human immunodeficiency virus
QPKTAGTNC

SEQ ID NO: 16 - protein, 9 aa, Human immunodeficiency virus
SFITKALGI

SEQ ID NO: 17 - protein, 9 aa, Human immunodeficiency virus
KKAKGWMYK

SEQ ID NO: 18 - protein, 9 aa, Human immunodeficiency virus
RCEYQAGHN

SEQ ID NO: 19 - protein, 9 aa, Human immunodeficiency virus
QYLALAALI

SEQ ID NO: 20 - protein, 9 aa, Human immunodeficiency virus
AGHNKVGSL

SEQ ID NO: 21 - protein, 9 aa, Human immunodeficiency virus
KPQKTKGHR

SEQ ID NO: 22 - protein, 9 aa, Human immunodeficiency virus
WAGVEAIIR

SEQ ID NO: 23 - protein, 9 aa, Human immunodeficiency virus
TYGDTWAGV

SEQ ID NO: 24 - protein, 9 aa, Human immunodeficiency virus
AGVEAIIRI

SEQ ID NO: 25 - protein, 9 aa, Human immunodeficiency virus
IVLIEYRKI

SEQ ID NO: 26 - protein, 9 aa, Human immunodeficiency virus
EEALAALVD

SEQ ID NO: 27 - protein, 9 aa, Human immunodeficiency virus
TQPIPIVAI

SEQ ID NO: 28 - protein, 9 aa, Human immunodeficiency virus
VLIEYRKIL

SEQ ID NO: 29 - protein, 9 aa, Human immunodeficiency virus
RQARRNRRR

SEQ ID NO: 30 - protein, 9 aa, Human immunodeficiency virus
SPQILVESP

SEQ ID NO: 31 - protein, 9 aa, Human immunodeficiency virus
SGDSDEELI

SEQ ID NO: 32 - protein, 9 aa, Human immunodeficiency virus
LPPLERLTL